(19) United States
(12) Patent Application Publication
(10) Pub. No.: US 2016/0026766 A1
(43) Pub. Date: Jan. 28, 2016

(54) Methods for Doing Business Using Biomarkers

(71) Applicant: Klaritos, Inc., Menlo Park, CA (US)

(72) Inventor: Vijay Ramakrishnan, Menlo Park, CA (US)

(21) Appl. No.: 14/877,822

(22) Filed: Oct. 7, 2015

Related U.S. Application Data
(63) Continuation of application No. 12/742,056, filed on Aug. 19, 2011, now abandoned.
(60) Provisional application No. 60/986,548, filed on Nov. 8, 2007.
(60) Provisional application No. 60/986,547, filed on Nov. 8, 2007.
(60) Provisional application No. 60/986,546, filed on Nov. 8, 2007.
(60) Provisional application No. 60/986,545, filed on Nov. 8, 2007.

(51) Int. Cl. G06F 19/00; G06Q 50/22; C12Q 1/68; G06Q 30/02

(52) U.S. Cl. CPC G06F 19/345; G06Q 30/0251; G06F 19/363; G06Q 50/22; C12Q 1/6883; C12Q 2600/106; C12Q 2600/158

(57) ABSTRACT

The present disclosure relates generally to methods for doing business by using one or more biomarkers (e.g., a subject's genetic polymorphisms) to improve the research, development, testing, commercialization and/or marketing of a drug by identifying those subjects for whom the particular drug is likely to be more effective.

### FIELD

The present disclosure relates to methods for doing business by using one or more biomarkers (e.g., a subject's genetic polymorphisms) to improve the research, development, testing, regulatory approval, commercialization, marketing, sales or use of a drug by identifying those subjects for whom the particular drug is likely to be more effective.

### BACKGROUND

The research and development process for a new drug generally requires that several milestones be reached before it is given FDA approval so that it can be marketed and eventually sold to a consumer. Any event that delays this process may cost the affected company a loss of revenue in the billions of dollars annually and delay the availability of life-saving drugs. Conversely, any change that can accelerate the commercialization and/or development of a potential drug can bring significant financial benefits and advance life-saving drugs to market.

Drugs (e.g., pharmaceuticals, biologics, therapeutic antibodies, etc.) are typically developed to interact with a single version of a gene product (e.g., protein or receptor). Therefore, a drug may only be effective in individuals that have a particular genetic variation of the gene product for which the drug was designed. As such, individuals who have a genetic variation for which the drug was not designed, may not respond to the drug and/or may experience adverse side effects (e.g., increased toxicity). For example, a number of antibodies have been developed for their use in therapies for a variety of diseases, disorders or conditions. However, it is generally acknowledged that most of these antibodies (e.g., Rituximab) work more effectively for some patients than others.

Given that patients selected from the general population respond differently to a drug, companies (e.g., pharmaceutical and biotechnology) may unnecessarily discontinue further drug development after an unsuccessful trial, fail to obtain regulatory approvals for promising drug candidates, or, even if approvals are obtained, be unable to market an approved drug effectively to those whom are likely to benefit from the drug.

### SUMMARY

The present disclosure relates generally to methods for doing business by using one or more biomarkers (e.g., a subject's genetic polymorphisms) to improve the research, development, testing, regulatory approval, commercialization, marketing, sales or use of a drug by identifying those subjects for whom the particular drug is likely to be more effective.

The present disclosure also relates generally to methods for doing business by using one or more biomarkers (e.g., a subject's genetic polymorphisms) to improve the research and development of a drug by directing and focusing efforts on those subjects for whom a particular drug is likely to be more effective.

The present disclosure also relates generally to methods for doing business by using one or more biomarkers (e.g., a subject's genetic polymorphisms) to customize a therapy to be specifically marketed to a subject for the treatment of a disease or disorder.

The present disclosure also relates generally to methods for doing business by using one or more biomarkers (e.g., a subject's genetic polymorphisms) to determine those subjects for which a particular drug is likely to be effective and/or ineffective, thereby gathering information about the drug and selling the information regarding the drug to one or more parties. Such information may be used, for example, in stratified medicine to associate more than one patient with a particular therapy.

The present disclosure relates generally to methods for doing business by using one or more biomarkers (e.g., a subject's genetic polymorphisms) to improve the treatment of subjects by identifying those subjects for whom the particular drug is likely to be more effective and administering the drug to the selected subjects.

A population may be categorized into groups based upon its FcγRIIa polymorphism, FcγRIIIa polymorphism and FcγRIIb polymorphism. Given that each group may respond differently to a therapy (e.g., therapeutic antibody), a business may selectively target those subjects that will exhibit an optimal response to the therapy. For example, the determination of a subject's FcγRIIa polymorphism, FcγRIIIa polymorphism or FcγRIIb polymorphism, or an FcγRIIa polymorphism, FcγRIIIa polymorphism and FcγRIIb polymorphism (e.g., FcγRIIIa V/F¹⁵⁸, FcγRIIa H/R¹³¹ and FcγRIIb 2B.1/2B.4 polymorphisms) may be used to stratify a patient population into categories (e.g., more than three categories) associated with response to a therapy (e.g., an antibody therapy) for an ADCC-treatable disease or disorder (including, e.g., a neoplastic disease, an autoimmune disease, a microbial infection, or an allograft rejection).

The present disclosure provides methods to improve the marketing of a therapy (e.g., therapeutic antibody) to be placed on the market (or already on the market) by selecting one or more therapies for a subject's (or population's) particular genotype. A focused marketing approach for a therapy (including, e.g., a therapeutic antibody) to a portion of the population with a specified FcγRIIa polymorphism, FcγRIIIa polymorphism and/or FcγRIIb polymorphism (e.g., a FcγRIIa H/R¹³¹, FcγRIIIa V/F¹⁵⁸ and/or a FcγRIIb 2B.1/2B.4 polymorphism) may reduce the expenses associated with poor responsiveness, time required for treatment and the need for additional therapies.

The present disclosure also provides methods for doing business by using one or more biomarkers (e.g., diagnostics) to direct and focus research/development of a therapy (e.g., a therapeutic antibody). An antibody development process may be directed and focused by, for example, making one or more changes in the manner in which clinical trials are conducted to reduce the time and/or expense for development. These methods may rely upon a determination of a FcγRIIa polymorphism, a FcγRIIIa polymorphism and/or FcγRIIb (e.g., a FcγRIIa H/R¹³¹, FcγRIIIa V/F¹⁵⁸ and/or a FcγRIIb 2B.1/2B.4 polymorphism) in the subject population that exhibits an optimal response to the therapy (e.g. antibody therapy). Subjects that have a genotype which indicates they are likely to be responsive to a specific antibody therapy may be chosen for clinical trials (e.g., phase I-IV) with the antibody. Such a targeted selection of subjects may lower development costs by dramatically reducing the sizes of required clinical trials and/or by decreasing the overall number of clinical trials, thereby facilitating quick regulatory approval and advancing the therapeutic antibody to market.

The present disclosure also relates generally to methods for doing business by using one or more biomarkers (e.g., a subject's genetic polymorphisms) to develop/customize a therapy for the treatment of a disease or disorder in a subject. For example, a therapy may be developed/customized for a subject based on their FcγRIIa polymorphism, FcγRIIIa polymorphism and/or FcγRIIb polymorphism (e.g., a FcγRIIa H/R¹³¹, FcγRIIIa V/F¹⁵⁸ and/or a FcγRIIb 2B.1/2B.4 polymorphism).

Methods are provided for conducting a business by selecting an antibody with an optimal effector function (e.g., ADCC activity) for a patient group with a FcγRIIIa V/V¹⁵⁸ and FcγRIIa H/H¹³¹ genotype, a FcγRIIIa V/F¹⁵⁸ and FcγRIIa H/H¹³¹ genotype, a FcγRIIIa F/F¹⁵⁸ and FcγRIIa H/H¹³¹ genotype, a FcγRIIIa V/V¹⁵⁸ and FcγRIIa H/R¹³¹ genotype, a FcγRIIIa V/F¹⁵⁸ and FcγRIIa H/R¹³¹ genotype, a FcγRIIIa F/F¹⁵⁸ and FcγRIIa H/R¹³¹ genotype, a FcγRIIIa V/V¹⁵⁸ and FcγRIIa R/R¹³¹ genotype, a FcγRIIIa V/F¹⁵⁸ and FcγRIIa R/R¹³¹ genotype, or a FcγRIIIa F/F¹⁵⁸ and FcγRIIa R/R¹³¹ genotype and marketing the antibody to health care providers, including physicians, nurses, hospitals and medical insurance providers and/or heath care consumers including, patients with a disease or disorder for administration to a patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for doing business by using a determination of a patient's FcγRIIIa V/V¹⁵⁸ and FcγRIIa H/H¹³¹ genotype, FcγRIIIa V/F¹⁵⁸ and FcγRIIa H/H¹³¹ genotype, FcγRIIIa F/F¹⁵⁸ and FcγRIIa H/H¹³¹ genotype, FcγRIIIa V/V¹⁵⁸ and FcγRIIa H/R¹³¹ genotype, FcγRIIIa V/F¹⁵⁸ and FcγRIIa H/R¹³¹ genotype, FcγRIIIa F/F¹⁵⁸ and FcγRIIa H/R¹³¹ genotype, FcγRIIIa V/V¹⁵⁸ and FcγRIIa R/R¹³¹ genotype, FcγRIIIa V/F¹⁵⁸ and FcγRIIa R/R¹³¹ genotype, or FcγRIIIa F/F¹⁵⁸ and FcγRIIa R/R¹³¹ genotype to select an antibody for treatment of a disease or disorder and marketing the antibody to health care providers, including physicians, nurses, hospitals and medical insurance providers and/or heath care consumers including, patients with a disease or disorder for administration to the patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient has an FcγRIIa H/H¹³¹ genotype, an FcγRIIa H/R¹³¹ genotype or an FcγRIIa R/R¹³¹ genotype; selecting the patient with the V/V¹⁵⁸ genotype and the H/H¹³¹ genotype for treatment with the antibody and marketing the antibody to health care providers, including physicians, nurses, hospitals and medical insurance providers and/or heath care consumers including, patients with a disease or disorder for administration to the patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient has an FcγRIIa H/H¹³¹ genotype, an FcγRIIa H/R¹³¹ genotype or an FcγRIIa R/R¹³¹ genotype; selecting the patient with the V/F¹⁵⁸ genotype and the H/H¹³¹ genotype for treatment with the antibody and marketing the antibody to health care providers, including physicians, nurses, hospitals and medical insurance providers and/or heath care consumers including, patients with a disease or disorder for administration to the patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient has an FcγRIIa H/H¹³¹ genotype, an FcγRIIa H/R¹³¹ genotype or an FcγRIIa R/R¹³¹ genotype; selecting the patient with the F/F¹⁵⁸ genotype and the H/H¹³¹ genotype for treatment with the antibody and marketing the antibody to health care providers, including physicians, nurses, hospitals and medical insurance providers and/or heath care consumers including, patients with a disease or disorder for administration to the patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient has an FcγRIIa H/H¹³¹ genotype, an FcγRIIa H/R¹³¹ genotype or an FcγRIIa R/R¹³¹ genotype; selecting the patient with the V/V¹⁵⁸ genotype and the H/R¹³¹ genotype for treatment with the antibody and marketing the antibody to health care providers, including physicians, nurses, hospitals and medical insurance providers and/or heath care consumers including, patients with a disease or disorder for administration to the patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient has an FcγRIIa H/H¹³¹ genotype, an FcγRIIa H/R¹³¹ genotype or an FcγRIIa R/R¹³¹ genotype; selecting the patient with the V/F¹⁵⁸ genotype and the H/R¹³¹ genotype for treatment with the antibody and marketing the antibody to health care providers, including physicians, nurses, hospitals and medical insurance providers and/or heath care consumers including, patients with a disease or disorder for administration to the patient/patient group. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient has an FcγRIIa H/H¹³¹ genotype, an FcγRIIa H/R¹³¹ genotype or an FcγRIIa R/R¹³¹ genotype; selecting the patient with the F/F¹⁵⁸ genotype and the H/R¹³¹ genotype for treatment with the antibody and marketing the antibody to health care providers, including physicians, nurses, hospitals and medical insurance providers and/or heath care consumers including, patients with a disease or disorder for administration to the patient group/patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient has an FcγRIIa H/H¹³¹ genotype, an FcγRIIa H/R¹³¹ genotype or an FcγRIIa R/R¹³¹ genotype; selecting the patient with the V/V¹⁵⁸ genotype and the R/R¹³¹ genotype for treatment with the antibody and marketing the antibody to health care providers, including physicians, nurses, hospitals and medical insurance providers and/or heath care consumers including, patients with a disease or disorder for administration to the patient group/patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient, has an FcγRIIa H/H¹³¹ genotype, an FcγRIIa H/R¹³¹ genotype or an FcγRIIa R/R¹³¹ genotype; selecting the patient with the V/F¹⁵⁸ genotype and the R/R¹³¹ genotype for treatment with the antibody and marketing the antibody to health care providers, including physicians, nurses, hospitals and medical insurance providers and/or heath care consumers including, patients with a disease or disorder for administration to the patient group/patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for conducting a business by determining if a patient has an FcγRIIIa V/V¹⁵⁸ genotype, an FcγRIIIa V/F¹⁵⁸ or an FcγRIIIa F/F¹⁵⁸ genotype; determining if the patient has an FcγRIIa H/H¹³¹ genotype, an FcγRIIa H/R¹³¹ genotype or an FcγRIIa R/R¹³¹ genotype; selecting the patient with the F/F¹⁵⁸ genotype and the R/R¹³¹ genotype for treatment with the antibody and marketing the antibody to health care providers, including physicians, nurses, hospitals and medical insurance providers and/or heath care consumers including, patients with a disease or disorder for administration to the patient group/patient. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are provided for selecting subjects for a clinical trial by administering an antibody to a population of subjects, determining the FcγRIIIa¹⁵⁸ and FcγRIIa¹³¹ genotype of the subjects with an optimal level of response to the antibody and enrolling subjects with the above-selected genotype in clinical trials with the antibody. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient, group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

Methods are also provided for generating, including increasing, revenue by optimizing a therapeutic antibody used to treat subjects with a FcγRIIIa V/V¹⁵⁸ and FcγRIIa H/H¹³¹ genotype, a FcγRIIIa V/F¹⁵⁸ and FcγRIIa H/H¹³¹ genotype, a FcγRIIIa F/F¹⁵⁸ and FcγRIIa H/H¹³¹ genotype, a FcγRIIIa V/V¹⁵⁸ and FcγRIIa H/R¹³¹ genotype, a FcγRIIIa V/F¹⁵⁸ and FcγRIIa H/R¹³¹ genotype, a FcγRIIIa F/F¹⁵⁸ and FcγRIIa H/R¹³¹ genotype, a FcγRIIIa V/V¹⁵⁸ and FcγRIIa R/R¹³¹ genotype, a FcγRIIIa V/F¹⁵⁸ and FcγRIIa R/R¹³¹ genotype, or a FcγRIIIa F/F¹⁵⁸ and FcγRIIa R/R¹³¹ genotype and marketing the optimized antibody to health care providers, including physicians, nurses, hospitals and medical insurance providers and/or heath care consumers including, patients with a disease or disorder with the above-selected genotype. Optionally, and in addition to the FcγRIIIa and FcγRIIa selection as described above, the antibody may be further selected based upon the FcγRIIb 2B.1/2B.1, 2B.1/2B.4 or 2B.4/2B.4 genotype of the patient group. As an alternative to the FcγRIIIa and FcγRIIa selection described above, the antibody may be selected for a patient group on the basis of their FcγRIIa (e.g., H/H¹³¹, H/R¹³¹ or R/R¹³¹) and FcγRIIb (e.g., 2B.1/2B.1, 2B.1/2B.4 or 2B.4/2B.4) genotype.

In some embodiments, the optimizing comprises altering the Fc portion of the antibody, to a Fc region associated with one or more optimal effector functions for the genotype.

In some embodiments of the disclosure, the antibody may be any antibody used to treat a subject, including for example, a monoclonal antibody. In some embodiments, the monoclonal antibody is Rituximab (RITUXIN®). In other embodiments the monoclonal antibody Alemtuzumab (CAMPATH®), Daclizumab (ZENAPAX®), Trastuzumab (HERCEPTIN®), Omalizumab (XOLAIR®), Efalizumab (RAPTIVA®), Bevacizumab (AVASTIN®), Infliximab (REMICADE®), Adalimumab (HUMIRA®), Cetuximab (ERBITUX®), Basiliximab (SIMULECT®), Palivizumab (SYNAGIS®), Panitumumab (VECTIBIX®), Natalizumab (TYSABRI®), Gemtuzumab (MYLOTARG®), Abciximab (REOPRO®), Ranibizumab (LUCENTIS®), Eculizumab (SOLIRIS®), Muromonab (OKT3®), Tositumomab (BEXXAR®) or Ibritumomab (ZEVALIN®).

In some embodiments of the disclosure, the antibody is a polyclonal antibody or a Fc fusion protein.

In some embodiments of the disclosure, the disease or disorder is selected from a neoplastic disease, an autoimmune disease, a microbial infection, and allograft rejection. In other embodiments, the neoplastic disease is non-Hodgkin's lymphoma (NHL). In some embodiments, the NHL is follicular lymphoma.

Additional features and advantages are described herein, and will be apparent from, the following Detailed Description.

### DETAILED DESCRIPTION

The present disclosure provides methods for doing business by using one or more biomarkers (e.g., a subject's FcγRIIa polymorphism, FcγRIIIa polymorphism and/or FcγRIIb polymorphism) to improve the research, development, testing, regulatory approval, commercialization, marketing, sales or use of drugs by identifying those patients for whom particular drugs are likely to be more effective.

Generally methods are provided for doing business by using one or more biomarkers (e.g., a FcγRIIIa V/F¹⁵⁸, a FcγRIIa H/R¹³¹ and/or a FcγRIIb 2B.1/2B.4 polymorphism) to select a therapeutic antibody for the treatment of a disease or disorder. The therapeutic antibody may then be marketed to health care providers, including physicians, nurses, hospitals and medical insurance providers and/or heath care consumers including, patients with a disease or disorder, for administration to the patient.

Further, methods are provided for conducting a business by using one or more biomarkers (e.g., a subject's genetic polymorphisms) to make informed decisions on whether or not to continue clinical trials (e.g., phase I to IV), enter later-phases of trials and/or select which subjects to enroll in later-stage trials (e.g., phase III or IV). For example, subject's that are predicted to exhibit a response to a therapeutic antibody based on their genotype may be selected for clinical trials thereby increasing the likelihood that the drug will be approved.

The present disclosure also provides methods for generating, including increasing, revenue by optimizing one or more therapeutic antibodies for a particular FcγRIIa, FcγRIIIa and/or FcγRIIb genotype (e.g., a FcγRIIa H/R¹³¹, FcγRIIIa V/F¹⁵⁸ and/or FcγRIIB polymorphism) and marketing the optimized antibody to subjects exhibiting the particular FcγRIIa, FcγRIIIa and/or FcγRIIb genotype. For example, a business may use Fe regions that have been optimized for a particular FcγRIIa, FcγRIIIa and/or FcγRIIb genotype to transfer the optimized properties to other antibodies.

Although any methods and materials similar or equivalent to those described herein can be used in the practice or testing of the present disclosure, the preferred methods and materials are described.

### Methods for Determining a Subject's Genotype

A subject's polymorphism at a particular genetic loci (e.g., a FcγRIIa H/R¹³¹, FcγRIIIa V/F¹⁵⁸ and/or a FcγRIIb 2B.1/2B.4 polymorphism) may be determined at the protein level (e.g., by assaying for a polymorphic protein) and/or at the nucleic acid level (e.g., by assaying for the presence of a nucleic acid polymorphism).

Any biological sample that comprises a polynucleotide from a subject may be suitable for use in the methods of the disclosure. The biological sample may be processed so as to isolate the polynucleotide. Alternatively, whole cells or other biological samples may be used without isolation of the polynucleotides contained therein. Detection of a polymorphism in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; and/or mismatch cleavage detection. Detection of a polymorphism can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; and/or an alteration in corresponding polypeptide activity.

Detection of a polymorphism by analyzing a polynucleotide sample can be conducted in a number of ways. For example, a nucleic acid sample can be amplified with primers which amplify a region known to comprise the genetic polymorphism(s). Additionally or alternatively, the region of interest can be cloned into a suitable vector and grown in sufficient quantity for analysis. The nucleic acid may be amplified by conventional techniques, such as a polymerase chain reaction (PCR), to provide sufficient amounts for analysis (see, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press). Once the region comprising a polymorphism has been amplified, the polymorphism can be detected in the PCR product by nucleotide sequencing (e.g., SSCP analysis). PCR may be used to determine whether a polymorphism is present by using a primer that is specific for the polymorphism. Such methods may comprise the steps of collecting from an individual a biological sample comprising the subject's genetic material as a template, optionally isolating template nucleic acid (genomic DNA, mRNA, or both) from the biological sample, contacting the template nucleic acid sample with one or more primers that specifically hybridize with a target polymorphic nucleic acid molecule under conditions such that hybridization and amplification of the template nucleic acid molecules in the sample occurs, and detecting the presence, absence, and/or relative amount of an amplification product and comparing the length to a control sample. Observation of an amplification product of the expected size is an indication that the polymorphism contained within the polymorphic primer is present in the nucleic acid sample. Parameters such as hybridization conditions, polymorphic primer length, and position of the polymorphism within the polymorphic primer may be chosen such that hybridization will not occur unless a polymorphism present in the primer(s) is also present in the sample nucleic acid (see, e.g., Saiki et al. (1986) Nature 324:163; and Saiki et al (1989) Proc. Natl. Acad. Sci. USA 86:6230).

Alternatively, various methods are known in the art that utilize oligonucleotide ligation as a means of detecting polymorphisms (see, e.g., Riley et al. (1990) Nucleic Acids Res. 18:2887-2890; and Delahunty et al., (1996) Am. J. Hum. Genet. 58:1239-1246).

Detectable labels may be included in the amplification reactions described herein. Suitable labels may include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid may be sequenced by a dideoxy chain termination method or other well-known methods. A variety of sequencing reactions known in the art can be used to directly sequence the relevant gene, or a portion thereof in which a specific polymorphism is known to occur, and detect polymorphisms by comparing the sequence of the sample nucleic acid with a reference polynucleotide that contains the polymorphism. Any of a variety of automated sequencing procedures can be used (see, e.g., WO 94/16101; Cohen et al. (1996) Adv. Chromatography 36:127-162).

Hybridization with the variant sequence may also be used to determine the presence of a polymorphism. Hybridization analysis can be carried out in a number of different ways, including, but not limited to Southern blots, Northern blots, dot blots and microarrays. The hybridization pattern of a control and variant sequence to an array of oligonucleotide probes immobilized on a solid support, e.g., as described in U.S. Pat. No. 5,445,934, or in WO 95/35505, may also be used as a means of detecting the presence of variant sequences. Identification of a polymorphism in a nucleic acid sample can be performed by hybridizing a sample and control nucleic acids to high density arrays containing hundreds or thousands of oligonucleotide probes (see, e.g., Cronin et al., (1996) Human Mutation 7:244-255; and Kozal et al., (1996) Nature Med. 2:753-759).

Single strand conformational polymorphism (SSCP) analysis; denaturing gradient gel electrophoresis (DGGE); mismatch cleavage detection; and heteroduplex analysis in gel matrices can also be used to detect polymorphisms. Alternatively, where a polymorphism creates or destroys a recognition site for a restriction endonuclease (restriction fragment length polymorphism, RFLP), the sample may be digested with that endonuclease, and the products size fractionated to determine whether the fragment was digested. Fractionation may be performed by gel or capillary electrophoresis, particularly acrylamide or agarose gels (see, e.g., “Laboratory Methods for the Detection of Mutations and Polymorphisms in DNA” (1997) G. R. Taylor, ed., CRC Press).

A number of methods are available for determining the expression of a polymorphic nucleic acid molecule, e.g., a polymorphic mRNA, or polymorphic polypeptide in a particular sample. Diagnosis may be performed by a number of methods to determine the absence or presence or altered amounts of normal or abnormal mRNA in a patient sample. For example, detection may utilize staining of cells or histological sections with labeled antibodies, performed in accordance with conventional methods. Cells may be permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including for example, flow cytometry of dissociated cells, microscopy, radiography and scintillation counting.

Alternatively, one may focus on the expression of mRNA. Biochemical studies may be performed to determine whether a sequence polymorphism in a coding region or control regions may be associated with disease. Disease associated polymorphisms may include deletion or truncation of the gene, mutations that alter expression level, that affect the activity of the protein.

Screening for mutations in a polymorphic polypeptide may be based on the functional or antigenic characteristics of the protein. Protein truncation assays are useful in detecting deletions that may affect the biological activity of the protein. Various immunoassays designed to detect polymorphisms in polymorphic polypeptides may be used in screening. The activity of the encoded a polymorphic polypeptide may be determined by comparison with a reference polypeptide lacking a specific polymorphism.

Diagnostic methods of the subject disclosure in which the level of polymorphic gene expression may be of interest will typically involve comparison of the relevant nucleic acid abundance of a sample of interest with that of a control value to determine any relative differences, where the difference may be measured qualitatively and/or quantitatively, which differences are then related to the presence or absence of an abnormal gene expression pattern. A variety of different methods for determining the nucleic acid abundance in a sample (see, e.g., Pietu et al., Genome Res. (June 1996) 6: 492-503; Zhao et al., Gene (Apr. 24, 1995) 156: 207-213; Soares, Curr. Opin. Biotechnol. (October 1997) 8: 542-546; Raval, J. Pharmacol. Toxicol. Methods (November 1994) 32: 125-127; Chalifour et al., Anal. Biochem. (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298).

In practicing the methods, the sample obtained from the host may be assayed to determine the genotype of the host or subject from which the sample was obtained with respect to at least one, i.e., one or more, polymorphisms. In certain embodiments, the at least one polymorphism is an FcγR polymorphism. An FcγR polymorphism is a polymorphism present in an FcγR (Fe receptor) protein. FcγR proteins of interest include, but are not limited to, FcγRII proteins (e.g., FcγRIIB, FcγRIIA, also known as CD32 (whose amino acid and nucleotide sequence is present at Genbank accession nos. NM_{—}021642 or M28697)); FcγRIII proteins (e.g., FcγRIIIA, also known as CD16 (whose amino acid and nucleotide sequence is present at Genbank accession nos. BC036723; BC033678; BC017865 and NM_{—}000569)), proteins and the like. In certain embodiments, the sample is assayed to determine the genotype of the host with respect to a single target polymorphism, where in these embodiments, the single polymorphism is an FcγRII polymorphism, such as an FcγRIIA polymorphism, where a specific representative FcγRIIA polymorphism of interest is the FcγRIIa H/R¹³¹ polymorphism (where the nucleotide codons encoding the H and R residues of the polymorphism are CAT and CGT, respectively). In certain embodiments, the sample is assayed to determine the genotype of the host with respect to two or more different polymorphisms, where in these embodiments, the two or more different polymorphisms include at least one FcγR polymorphism. In certain of these embodiments, at least two of the polymorphisms are different FcγR polymorphisms, such as an FcγRII and an FcγRIII polymorphism. In certain embodiments, the sample is assayed for an FcγRII polymorphism, such as the specific FcγRIIa polymorphisms described above, an FcγRIII polymorphism, such as an FcγRIIIa polymorphism, including the FcγRIIIa V/F¹⁵⁸ polymorphism and/or a FcγRIIb polymorphism, including the FcγRIIb 2B.1/2B.4 polymorphism.

### Methods for the Selection of a Therapeutic Antibody for Treatment of a Subject

Methods are provided for selecting one or more therapeutic antibodies and/or antibody fragments for treatment of a subject (or population of subjects) based on the subject's (or population's) FcγRIIa, FcγRIIIa and/or FcγRIIb polymorphism. For example, an antibody therapy may be selected to treat a subject with a particular FcγRIIa (H/R¹³¹) polymorphism, FcγRIIIa (V/F¹⁵⁸) polymorphism and/or FcγRIIb (2B.1/2B.4) polymorphism.

A therapeutic antibody may be selected for a subject by genotyping the subject for a FcγRIIA polymorphism, a FcγRIIIA polymorphism and/or a FcγRIIB polymorphism and choosing an antibody predicted to exhibit an optimal response in the subject. In some embodiments, a subject's genotype may be compared with a reference chart and the predicted response determined from the reference chart.

TABLE A

| | FcγRIIIA V/V¹⁵⁸ | FcγRIIIA V/F¹⁵⁸ | FcγRIIIA F/F¹⁵⁸ |
|---|---|---|---|
| FcγRIIA | Group-I | Group-II | Group-III |
| H/H¹³¹ | V, V; H, H | V, F; H, H | F, F; H, H |
| FcγRIIA | Group-IV | Group-V | Group-VI |
| H/R¹³¹ | V, V; H, R | V, F; H, R | F, F; H, R |
| FcγRIIA | Group-VII | Group-VIII | Group-IX |
| R/R¹³¹ | V, V; R, R | V, F; R, R | F, F; R, R |

As an example, a reference chart with various combinations of FcγRIIA and FcγRIIIA polymorphisms, and corresponding categories of anticipated responsiveness to a monoclonal antibody therapy (e.g., Rituxan), are shown in Table A. Without being bound by a theory of the invention, the V¹⁵⁸ allele in FcγRIIIA may be a high-affinity/high-responder receptor while the F¹⁵⁸ allele may be a low-affinity/low-responder receptor. Similarly, without being bound by a theory of the invention, the H¹³¹ allele in FcγRIIA may be a high-affinity/high-responder receptor while the R¹³¹ allele may be a low-affinity/low-responder receptor.

In certain embodiments, the subject's genotype may be compared with a reference or control to make a diagnosis regarding the subject's predicted response to an antibody therapy.

Examples of genotypes associated with responsiveness to a therapeutic antibody, particularly Rituximab, include, but are not limited to: the FcγRIIA H/H¹³¹, FcγRIIIA V/V¹⁵⁸ genotype. Examples of genotypes associated with moderate responsiveness to a therapeutic antibody, particularly Rituximab, include the FcγRIIIa V/F¹⁵⁸, H/H¹³¹; the FcγRIIIa F/F¹⁵⁸, FcγRIIa H/H¹³¹; the FcγRIIIa V/V¹⁵⁸, FcγRIIa H/R¹³¹; and/or the FcγRIIIa V/V¹⁵⁸ FcγRIIa R/R¹³¹ genotype. Examples of genotypes associated with poor responsiveness to a therapeutic antibody, particularly Rituximab, include the FcγRIIIa V/F¹⁵⁸, FcγRIIa R/R¹³¹; the FcγRIIIa F/F¹⁵⁸, FcγRIIa R/R¹³¹; the FcγRIIIa V/F¹⁵⁸, FcγRIIa H/R¹³¹; and/or the FcγRIIIa F/F¹⁵⁸, FcγRIIa H/R¹³¹ genotype.

The predicted responsiveness of a subject to an antibody therapy may be categorized into different categories based on a FcγRIIA H/R¹³¹ polymorphism, FcγRIIIA V/F¹⁵⁸ polymorphism and/or FcγRIIB 2B.1/2B.4 polymorphism, e.g., more than three different categories, including, a) “high degree of responsiveness” b) “moderate degree of responsiveness” and c) “poor degree of responsiveness.”

Responsiveness may be determined at various times after treatment with a given antibody therapy, e.g., 1-3 months, 3-6 months, 6-9 months, or 9-12 months following treatment, e.g., following initiation of treatment. Thus, e.g., responsiveness can be expressed with a time component.

Responsiveness to an antibody therapy for a neoplastic disease may be determined by any assay known in the art, for example, antibody-dependent cell-mediated cytotoxicity (ADCC) response to tumor cells, reduction in tumor mass, and/or reduction in number of tumor cells. Responsiveness to an antibody therapy for an autoimmune disease may include a reduction in a symptom associated with the autoimmune disorder; reduction in the number and/or activity of an autoreactive B-cell; and/or reduction in the number and/or activity of an autoreactive T-cell. Responsiveness to an antibody therapy for allograft rejection can include one or more of: reduction in the amount of immunosuppressive drug that must be administered to an individual who is the recipient of an allograft and still maintain the allograft; duration of maintenance of the allograft; function of the allograft; reduction in the number and/or activity of alloreactive T-cells in the allograft recipient. Responsiveness to an antibody therapy for a viral infection can include one or more of: reduction in the number of viral genomes in a tissue, fluid, or other specimen from an individual; reduction in one or more symptoms of a viral infection Responsiveness can also be assessed using an in vitro ADCC assay.

A subject may be selected for treatment with an antibody by: (a) determining if the subject has an FcγRIIIA V/V¹⁵⁸ genotype, an FcγRIIIA V/F¹⁵⁸ genotypeor an FcγRIIIA F/F¹⁵⁸ genotype; (b) determining if the subject has an FcγRIIA H/H¹³¹ genotype, an FcγRIIA H/R¹³¹ genotype or an FcγRIIA R/R¹³¹ genotype; (c) optionally determining if the subject has an FcγRIIB 2B.1/2B.1 genotype, an FcγRIIB 2B.1/2B.4 or an FcγRIIB 2B.4/2B.4 genotype; (d) selecting the subject with a particular genotype for treatment with the antibody based on the genotype determination of steps (a), (b) and optionally (c); and (e) administering the antibody to the subject selected in step (d).

In some embodiments a subject may be selected with a FcγRIIIA V/V¹⁵⁸, FcγRIIA H/H¹³¹ genotype; a FcγRIIIA V/F¹⁵⁸, FcγRIIA H/H¹³¹ genotype; a FcγRIIIA F/F¹⁵⁸, FcγRIIA H/H¹³¹ genotype; a FcγRIIIA V/V¹⁵⁸, FcγRIIA H/R¹³¹ genotype; a FcγRIIIA V/F¹⁵⁸, FcγRIIA H/R¹³¹ genotype; a FcγRIIIA F/F¹⁵⁸, FcγRIIA H/R¹³¹ genotype; a FcγRIIIA V/V¹⁵⁸, FcγRIIA R/R¹³¹ genotype; a FcγRIIIA V/F¹⁵⁸, FcγRIIA R/R¹³¹ genotype; or a FcγRIIIA F/F¹⁵⁸, FcγRIIA R/R¹³¹ genotype.

In some embodiments, therapeutic antibody may be Rituximab (RITUXIN®), Alemtuzumab (CAMPATH®), Daclizumab (ZENAPAX®), Trastuzumab (HERCEPTIN®), Omalizumab (XOLAIR®), Efalizumab (RAPTIVA®), Bevacizumab (AVASTIN®), Infliximab (REMICADE®), Adalimumab (HUMIRA®), Cetuximab (ERBITUX®), Basiliximab (SIMULECT®), Palivizumab (SYNAGIS®), Panitumumab (VECTIBIX®), Natalizumab (TYSABRI®), Gemtuzumab (MYLOTARG®), Abciximab (REOPRO®), Ranibizumab (LUCENTIS®), Eculizumab (SOLIRIS®), Muromonab (OKT3®), Tositumomab (BEXXAR®) or Ibritumomab (ZEVALIN®).

Diseases and disorders that are treatable with an ADCC-based antibody therapy include, but are not limited to, neoplastic diseases; autoimmune diseases; allograft rejection, and microbial infections.

### Enhancement of Antibody Effector Functions

A therapeutic antibody may be optimized for a subject with a particular FcγRIIA and/or FcγRIIIA genotype by altering the Fc portion of the antibody to a sequence associated with optimal effector function (e.g., ADCC) in subjects with the genotype. For example, the Fc region from an antibody, including, for example, a variant antibody with one or more optimal effector functions for a particular FcγRIIA and FcγRIIIA genotype can be used to optimize one or more effector functions of other antibodies used to treat the same or other subjects with the FcγRIIA and FcγRIIIA genotype.

In some embodiments, the effector function is ADCC. In other embodiments, the effector function may be phagocytosis, opsonization, opsonophagocytosis, Clq binding, and/or complement dependent cell mediated cytotoxicity (CDC).

One or more effector functions of a therapeutic antibody used to treat a subject having an ADCC-treatable disease or disorder may be enhanced by genotyping the subject for an FcγRIIA polymorphism, a FcγRIIIA polymorphism and/or a FcγRIIB polymorphism, classifying the subject into one of more than three categories of ADCC activity for the antibody based on their FcγRIIA polymorphism, FcγRIIIA polymorphism and/or a FcγRIIB polymorphism and selecting an Fc nucleotide sequence that has at least one optimized effector function for the FcγRIIA polymorphism, the FcγRIIIA polymorphism and/or the FcγRIIB polymorphism, wherein at least one effector function of the antibody is enhanced by using the optimized Fc nucleotide sequence.

Fc cassettes may be optimized for the following FcγRIIA and FcγRIIIA genotypes, including: FcγRIIIA V/V¹⁵⁸, FcγRIIA H/H¹³¹; FcγRIIIA V/F¹⁵⁸, FcγRIIA H/H¹³¹; FcγRIIIA F/F¹⁵⁸, FcγRIIA H/H¹³¹; FcγRIIIA V/V¹⁵⁸, FcγRIIA H/R¹³¹; FcγRIIIA V/F¹⁵⁸, FcγRIIA H/R¹³¹; FcγRIIIA F/F¹⁵⁸, FcγRIIA H/R¹³¹; FcγRIIIA V/V¹⁵⁸, FcγRIIA R/R¹³¹; FcγRIIIA V/F¹⁵⁸, FcγRIIA R/R¹³¹; and/or FcγRIIIA F/F¹⁵⁸, FcγRIIA R/R¹³¹. Optionally, the Fc cassettes may be optimized for the following FcγRIIB genotypes, including FcγRIIB 2B.1/2B.1, FcγRIIB 2B.1/2B.4 and FcγRIIB 2B.4/2B.4.

Therapeutic antibodies used to treat a particular FcγRIIA (H/R¹³¹) genotype, FcγRIIIA (V/F¹⁵⁸) genotype and/or FcγRIIB (2B.1/2B.4) genotype may be modified to exhibit optimal ADCC activity. For instance the therapeutic antibody RITUXIN™ has optimal ADCC activity for subjects exhibiting a FcγRIIIA V/V¹⁵⁸, FcγRIIA H/H¹³¹ and/or FcγRIIB 2B.1/2B.1 genotype. Hence, the Fc nucleotide sequence of RITUXIN™ may be used to optimize ADCC activity of other antibodies used to treat subjects exhibiting a FcγRIIIA V/V¹⁵⁸, FcγRIIA H/H¹³¹ genotype. For instance, the Fc nucleotide sequence of RITUXIN™ may be used to replace the Fc nucleotide sequence present in ZENAPAX® to optimize its ADCC activity in subjects exhibiting a FcγRIIIA V/V¹⁵⁸, FcγRIIA H/H¹³¹ and/or FcγRIIB 2B.1/2B.1 genotype.

Examples of other therapeutic antibodies that can be engineered to have enhanced ADCC activity for a particular FcγRIIa and FcγRIIIa genotype include Rituximab (RITUXIN®), Alemtuzumab (CAMPATH®), Daclizumab (ZENAPAX®), Trastuzumab (HERCEPTIN®), Omalizumab (XOLAIR®), Efalizumab (RAPTIVA®), Bevacizumab (AVASTIN®), Infliximab (REMICADE®), Adalimumab (HUMIRA®), Cetuximab (ERBITUX®), Basiliximab (SIMULECT®), Palivizumab (SYNAGIS®), Panitumumab (VECTIBIX®), Natalizumab (TYSABRI®), Gemtuzumab (MYLOTARG®), Abciximab (REOPRO®), Ranibizumab (LUCENTIS®), Eculizumab (SOLIRIS®), Muromonab (OKT3®), Tositumomab (BEXXAR®) or Ibritumomab (ZEVALIN®).

Antibody effector functions, such as for example, ADCC, may be optimized for a particular FcγRIIA, FcγRIIIA and/or FcγRIIB genotype by altering the nucleotide sequence of the Fc portion of the antibody, to a Fc nucleotide sequence associated with optimal effector functions for the genotype. The nucleotide sequence of the Fc region of an antibody may be engineered by techniques commonly known in the art to derive the same nucleotide sequence of Fc that has optimized ADCC activity for a subject with a particular FcγRIIA, FcγRIIIA and/or FcγRIIB genotype.

In other embodiments, the ADCC activity of an antibody used to treat a subject with a particular FcγRIIA, FcγRIIIA and/or FcγRIIB genotype can be optimized by fusing a Fc nucleotide sequence from another antibody molecule which has optimized effector functions for the given genotype.

Alternatively, PCR amplification of gene fragments (i.e. the optimized Fc and antigen binding domain) can be carried out using anchor primers which give rise to complementary overhangs between two consecutive gene fragments which can subsequently be annealed and reamplified to generate a chimeric gene sequence (See, e.g., Current Protocols in Molecular Biology, Ausubel et al., eds., John Wiley & Sons, 1992).

Moreover, a nucleic acid encoding an antigen binding domain can be cloned into an expression vector containing an genotype optimized Fc region such that the antigen binding domain is linked in-frame to the optimized Fc region (see, e.g., U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367, 166; EP 394,827; International Publication Nos. WO 91/06570, WO 96/04388, WO 96/22024, WO 97/34631, and WO 99/04813; Ashkenazi et al., 1991, Proc. Natl. Acad. Sci. USA 88: 10535-10539; Traunecker et al., 1988, Nature, 331:84-86; Zheng et al., 1995, J. Immunol. 154:5590-5600; and Vil et al., 1992, Proc. Natl. Acad. Sci. USA 89:11337-11341).

The nucleotide sequences encoding a antigen binding domain and an Fc domain may be obtained from any information available to those of skill in the art (i.e., from Genbank, the literature, or by routine cloning) (See, e.g., Xiong et al., Science, 12; 294(554 1): 339-45 (2001)). The nucleotide sequence coding for an antibody fusion protein can be inserted into an appropriate expression vector, i.e., a vector that contains the necessary elements for the transcription and translation of the inserted protein-coding sequence. A variety of host-vector systems may be utilized in the present disclosure to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

### Business Methods

One or more biomarkers (e.g., a subject's genetic polymorphisms) may make it possible to improve the marketing process for a therapy (e.g., an antibody therapy) by tailoring business efforts to subjects most likely to exhibit a response to the therapy. In some embodiments, a biomarker may include a subject's FcγRIIA polymorphism, FcγRIIIA polymorphism and/or FcγRIIB polymorphism (e.g., FcγRIIIa V/F¹⁵⁸, FcγRIIa H/R¹³¹ and/or FcγRIM 2B.1/2B.4 polymorphisms).

The correlation between a subject's FcγRIIIa V/F¹⁵⁸, FcγRIIa H/R¹³¹ and/or FcγRIIB 2B.1/2B.4 genotype and response to a particular therapy (e.g., therapeutic antibody) may enable businesses to improve the marketing of therapies (e.g., therapeutic antibodies) by identifying segments of the population for whom particular therapies are likely to be more effective than other therapies. For example, subjects that are good responders to a therapeutic antibody (e.g., Rituximab) may be targeted by marketing efforts before subjects whom are moderate responders. Likewise, moderate responders to a therapeutic antibody may be targeted by marketing efforts before poor responders, whom may not be targeted at all. Alternatively, both the good and moderate responders to a therapeutic antibody (e.g., Rituximab) may be targeted by marketing efforts before subjects whom are poor responders.

Marketing efforts tailored for good responders for the therapeutic antibody may be similar or different from the marketing efforts tailored for the moderate and/or poor responders to the antibody. For example, a business may appropriate greater dollars for marketing to subjects considered good responders to the therapeutic antibody as compared to subjects whom are considered poor responders to the therapeutic antibody.

For example, subjects that may be good responders to an antibody therapy (e.g., Rituximab therapy) may be preferentially targeted by a business' marketing efforts. For example, subjects with a FcγRIIa H/H¹³¹, FcγRIIIa V/V¹⁵⁸ and/or FcγRIIB 2B.1/2B.1 genotype are generally good responders to Rituximab therapy and may be preferentially targeted by a business' marketing efforts. Further, subjects that present a FcγRIIIa V/F¹⁵⁸, H/H¹³¹; FcγRIIIa F/F¹⁵⁸, FcγRIIa H/H¹³¹; FcγRIIIa V/V¹⁵⁸, FcγRIIa H/R¹³¹; or FcγRIIIa V/V¹⁵⁸ FcγRIIa R/R¹³¹ genotype are moderate responders to Rituximab therapy and may not be targeted by a business' marketing efforts as much as the good responders. Subjects that exhibit a FcγRIIIa V/F¹⁵⁸, FcγRIIa R/R¹³¹; FcγRIIIa F/F¹⁵⁸, FcγRIIa R/R¹³¹; FcγRIIIa V/F¹⁵⁸, FcγRIIa H/R¹³¹, FcγRIIIa F/F¹⁵⁸, FcγRIIa H/R¹³¹ genotype are generally not good responders to Rituximab therapy and may not be targeted by business efforts (e.g., marketing) or may be targeted less than the good and moderate responders. Additionally or optionally, subjects that exhibit a FcγRIIB 2B.1/2B.1 genotype are generally good responders to Rituximab therapy and may be targeted by a business efforts, while subjects that exhibit a FcγRIIB 2B.4/2B.4 genotype are generally poor responders to Rituximab therapy and may not be targeted by business efforts.

Further, a business may use a diagnostic indicator to segregate a population that encounters negative side effects (or even toxicity) from a population that does not suffer negative effects. This may allow a company to effectively market its drug to subjects that do not experience any adverse side-effects and generate, including increase, revenues by keeping a therapeutic antibody on the market that would otherwise be withdrawn or to reintroduce a therapeutic antibody that has already been withdrawn due to adverse effects in some subjects. A large safety or efficacy differential and/or major improvement over the previous standard of care may permit a company to generate, including increase, revenues by charging a price premium to a patient group.

Marketing efforts for the therapy (e.g., therapeutic antibody) may consist of pricing, promotion and/or product placement. For example, a business may encourage physicians to preferentially prescribe patient specific therapies (e.g., therapeutic antibodies). Such marketing efforts to health care providers, including physicians, nurses, hospitals and medical insurance providers may comprise, for example, continuing medical education about the therapy (e.g., therapeutic antibody), advertisements about the therapy placed in peer-review journals, print/internet advertising or direct sale calls. Additionally or alternatively, marketing efforts may be directed to patients, including patients with a disease or disorder may include, for example, print, television, internet and/or radio advertisements.

Therapeutic antibodies specifically marketed to a population of subjects can generate, including increase, revenues by reducing the length of time it takes to advance a drug to market, reducing the cost of ineffective marketing (e.g., marketing the drug to patients which will not respond to the drug), and/or increasing the price premium for the therapeutic due to its high efficacy. Therapeutic antibodies that may be marketed according to the methods described herein include, for example, antibodies previously in-licensed, antibodies developed and/or commercialized independently using internal pharmaceutical development efforts and/or antibodies developed in collaboration with one or more partners. Additionally or alternatively, marketing efforts may be tailored to healthcare providers, insurance companies, government entities (e.g., Medicaid, Medicare) and/or employers or any other entity interested in achieving an economical and/or effective system for providing or paying for medical and/or life insurance. Such parties can utilize information concerning a patient's FcγRIIa H/R¹³¹, FcγRIIIa V/F¹⁵⁸ and/or FcγRIIB 2B.1/2B.4 genotype to selectively approve expensive therapeutic antibodies to patients whom exhibit a genotype correlated with good responsiveness to an antibody, evaluate better an individual's likelihood to suffer from disease prior to underwriting them and selecting more effective health and life insurance premiums for individuals.

A business may use one or more biomarkers (e.g., a subject's genetic polymorphisms) to direct and/or focus the research and development of a therapy (e.g., therapeutic antibody). Businesses may use a determination of a subject's genotype to make informed decisions on whether or not to continue clinical trials (e.g., phase I to IV), enter later-phases of trials or select which patients to enroll in later-stage trials (e.g., phase III or IV). For example, a biomarker may be used to enroll patients with a genetic predisposition for a positive response to a therapeutic antibody in clinical trials. Selective enrollment of subjects that are likely to respond to the drug (e.g., therapeutic antibody) can improve the therapeutic index for these subjects and improve the possibility of regulatory approval of the drug. Additionally, enrollment of subjects that are likely to respond to a drug in clinical trials may generate, including increase revenue, by decreasing the overall number of clinical trials required and spurring faster review cycles.

The first company to gain approval for their therapeutic antibody may be capable of enjoying a period of market exclusivity and be able to generate, including increase, revenues before any competitors advance their product to market. Additionally, a business that increases the probability that an antibody will elicit a good response in a subject limits the number of late-stage failures and unexpected late-stage delays (e.g., particularly in phase III clinical trials). Also, a business may obtain important intangible benefits from quick regulatory approval, including, for example, strengthened public trust and wider adoption due to superior clinical performance, investor credibility, stock market performance, research/development productivity gains, additional regulatory market protections (e.g., orphan drug status due to small population size) and/or a longer effective patent life arising from a shortened development time.

For example, subjects that may be good responders to an therapeutic antibody (e.g., Rituximab therapy) may be preferentially selected for clinical trials involving the therapeutic antibody. For example, subjects with a FcγRIIIa V/V¹⁵⁸ and FcγRIIa H/H¹³¹ genotype are generally good responders to Rituximab therapy and may be selected for clinical trials. Further, subjects that present a FcγRIIIa V/F¹⁵⁸, H/H¹³¹; FcγRIIIa F/F¹⁵⁸, FcγRIIa H/H¹³¹; FcγRIIIa V/V¹⁵⁸, FcγRIIa H/R¹³¹; or FcγRIIIa V/V¹⁵⁸ FcγRIIa R/R¹³¹ genotype are moderate responders to Rituximab therapy and may be less desirable for clinical trials than the good responders. Subjects that exhibit a FcγRIIIa V/F¹⁵⁸, FcγRIIa R/R¹³¹; FcγRIIIa F/F¹⁵⁸, FcγRIIa R/R¹³¹; FcγRIIIa V/F¹⁵⁸, FcγRIIa H/R¹³¹, FcγRIIIa F/F¹⁵⁸, FcγRIIa H/R¹³¹ genotype are generally poor responders to Rituximab therapy and may not be selected for clinical trials) or may be less desirable than the good and moderate responders. Additionally or optionally, subjects that exhibit a FcγRIIB 2B.1/2B.1 genotype are generally good responders to Rituximab therapy and may be preferentially selected for clinical trials, while subjects that exhibit a FcγRIIB 2B.4/2B.4 genotype are generally poor responders to Rituximab therapy and may not be selected for clinical trials.

Methods are also provided for generating, including increasing, revenue by optimizing one or more therapeutic antibodies for a particular FcγRIIA, FcγRIIIA and/or FcγRIIB genotype and marketing the optimized antibody to subjects with the particular FcγRIIA, FcγRIIIA and/or FcγRIIB genotype. For example, a therapeutic antibody may be tailored (e.g., by altering the nucleotide sequence of the Fc portion of the antibody, to a Fc nucleotide sequence associated with optimal effector functions for the genotype) for a subject that has a genotype associated with a moderate or poor response to an antibody therapy to achieve optimal response in the subject.

For example, Rituximab is associated with a moderate or poor response in subjects that exhibit a FcγRIIIa V/F¹⁵⁸, FcγRIIa H/H¹³¹; FcγRIIIa F/F¹⁵⁸, FcγRIIa H/H¹³¹; FcγRIIIa V/V¹⁵⁸, FcγRIIa H/R¹³¹; FcγRIIIa V/V¹⁵⁸ FcγRIIa R/R¹³¹ FcγRIIIa V/F¹⁵⁸, FcγRIIa R/R¹³¹; FcγRIIIa F/F¹⁵⁸, FcγRIIa R/R¹³¹; FcγRIIIa V/F¹⁵⁸, FcγRIIa H/R¹³¹; or a FcγRIIIa F/F¹⁵⁸, FcγRIIa H/R¹³¹ and/or a FcγRIIB 2B1/2B. or a 2B.4/2B.4 genotype. A business strategy may include modifying the Fc portion of Rituximab for each genotype, such that the modified antibody exhibits an optimal response in each subject. Modified therapeutic antibodies may be preferentially marketed to each genotype for which it was engineered thus generating, including increasing, revenues from antibodies that were originally not effective for subject's without the optimal FcγRIIa H/R¹³¹, FcγRIIIa V/F¹⁵⁸ and/or FcγRIIB 2B.1/2B.4 genotype.

Methods are also provided for doing business by using one or more biomarkers (e.g., a subject's genetic polymorphisms) to develop/customize a therapy which may be specifically marketed to the subject for the treatment of a disease or disorder. A diagnostic indictor may indicate that a subject is likely to respond to a particular drug, albeit not with an optimal response. Thus, the drug may be tailored for specifically for the subject to produce an optimal response. For example, a therapy may be developed/customized for a subject based on their FcγRIIA polymorphism, FcγRIIIA polymorphism and/or FcγRIIB polymorphism (e.g., FcγRIIIa V/F¹⁵⁸, FcγRIIa H/R¹³¹ and/or FcγRIIB 2B.1/2B.4 polymorphisms).

Methods are provided for doing business by using one or more biomarkers (e.g., a subject's polymorphisms) to determine those subjects for which a particular drug is likely to be effective and/or ineffective, thereby generating information about the drug and selling the information to one or more parties. For example, a business may be able to determine if a drug is likely to be effective for a subject based upon their FcγRIIA polymorphism, FcγRIIIA polymorphism and/or FcγRIIB (e.g., FcγRIIIa V/F¹⁵⁸, FcγRIIa H/R¹³¹ and/or FcγRIIB 2B.1/2B.4 polymorphisms). A business may generate, including increase, revenue by marketing and selling the information to other parties, including for example, the manufacturer and/or distributor of the drug, health care providers and/or patients.

For example, a business may determine the FcγRIIA polymorphism, FcγRIIIA polymorphism and/or FcγRIIB polymorphism (e.g., FcγRIIIa V/F¹⁵⁸, FcγRIIa H/R¹³¹ and/or FcγRIIB 2B.1/2B.4 polymorphisms) for subjects that exhibit a good, moderate and/or poor response to an antibody therapy and sell the information to other parties. Such information may be used, for example, in stratified medicine to associate one or more patients with a particular therapy.

Methods are provided for doing business by using one or more biomarkers (e.g., a subject's genetic polymorphisms) to improve the treatment of subjects by identifying those subjects for whom the particular drug is likely to be more effective and administering the drug to the selected subjects. For example, a health care provider, including a physician may use a biomarker to select a therapy that is predicted to elicit a good response in the treatment of a subject.

For example, a business may use a subject's FcγRIIA polymorphism, FcγRIIIA polymorphism and/or FcγRIIB polymorphism (e.g., FcγRIIIa V/F¹⁵⁸, FcγRIIa H/R¹³¹ and/or FcγRIIB 2B.1/2B.4 polymorphisms) as a biomarker to select a therapy that is predicted to elicit a good response in a subject. Such tailored therapy may generate, including increase revenues by strengthening the subject's confidence in the physician and creating greater public trust. Additionally, a business may generate, including increase, revenues from increased patient compliance with a therapeutic regimen (e.g., due to a good response to the therapy or through positive feedback from ongoing clinical testing that confirms the drug's efficacy/safety).

While the present disclosure has been described and illustrated herein by references to various specific materials, procedures and examples, it is understood that the disclosure is not restricted to the particular combinations of material and procedures selected for that purpose. This application claims the benefit of U.S. Patent Application 60/986,545, filed on Nov. 8, 2007; U.S. Patent Application 60/986,546, filed on Nov. 8, 2007; U.S. Patent Application 60/986,547, filed on Nov. 8, 2007; and U.S. Patent Application 60/986,548, filed on Nov. 8, 2007, the entire contents of each of which are incorporated herein by reference.

1. A method for conducting a business, said method comprising:
(a) selecting an antibody useful for treatment of a disease or disorder with optimal ADCC activity for a patient group with a FcγRIIIa V/V¹⁵⁸, FcγRIIa H/H¹³¹; FcγRIIIa V/F¹⁵⁸, FcγRIIa H/H¹³¹; FcγRIIIa F/F¹⁵⁸, FcγRIIa H/H¹³¹; FcγRIIIa V/V¹⁵⁸, FcγRIIa H/R¹³¹; FcγRIIIa V/V¹⁵⁸, FcγRIIa R/R¹³¹; FcγRIIIa V/F¹⁵⁸, FcγRIIa R/R¹³¹; FcγRIIIa F/F¹⁵⁸, FcγRIIa R/R¹³¹; FcγRIIIa V/F¹⁵⁸, FcγRIIa H/R¹³¹; or a FcγRIIIa F/F¹⁵⁸, FcγRIIa H/R¹³¹ genotype; and
(b) marketing the antibody to health care providers or health care consumers for administration to the patient group.

2. The method of claim 1, wherein the disease or disorder is selected from a neoplastic disease, an autoimmune disease, a microbial infection, and allograft rejection.

3. The method of claim 2, wherein the neoplastic disease is non-Hodgkin's lymphoma (NHL).

4. The method of claim 3, wherein the NHL is follicular lymphoma.

5. The method of claim 1, wherein the antibody is a monoclonal antibody, polyclonal antibody or an Fc fusion protein.

6-7. (canceled)

8. The method of claim 1, wherein the marketing is accomplished by continuing medical education, peer-review journals, Internet, print advertising or direct sale calls.

9. The method of claim 1, wherein the health care providers are selected from the group consisting of physicians, nurses, hospitals and medical insurance providers.

10. (canceled)

11. A method for conducting a business, said method comprising:
(a) determining if a patient has an FcγRIIIa V/V¹⁵⁸ genotype, an FcγRIIIa V/F¹⁵⁸ or an FcγRIIIa F/F¹⁵⁸ genotype;
(b) determining if the patient has an FcγRIIa H/H¹³¹ genotype, an FcγRIIa H/R¹³¹ genotype or an FcγRIIa R/R¹³¹ genotype;
(c) selecting the patient with the V/V¹⁵⁸ genotype and the H/H¹³¹ genotype for treatment with the antibody based on the genotype determination of steps (a) and (b), selecting the patient with the V/F¹⁵⁸ genotype and the H/H¹³¹ genotype for treatment with the antibody based on the genotype determination of steps (a) and (b), selecting the patient with the F/F¹⁵⁸ genotype and the H/H¹³¹ genotype for treatment with the antibody based on the genotype determination of steps (a) and (b), selecting the patient with the V/V¹⁵⁸ genotype and the H/R¹³¹ genotype for treatment with the antibody based on the genotype determination of steps (a) and (b), selecting the patient with the V/F¹⁵⁸ genotype and the H/R¹³¹ genotype for treatment with the antibody based on the genotype determination of steps (a) and (b), selecting the patient with the F/F¹⁵⁸ genotype and the H/R¹³¹ genotype for treatment with the antibody based on the genotype determination of steps (a) and (b), selecting the patient with the V/V¹⁵⁸ genotype and the R/R¹³¹ genotype for treatment with the antibody based on the genotype determination of steps (a) and (b), selecting the patient with the V/F¹⁵⁸ genotype and the R/R¹³¹ genotype for treatment with the antibody based on the genotype determination of steps (a) and (b), or selecting the patient with the F/F¹⁵⁸ genotype and the R/R¹³¹ genotype for treatment with the antibody based on the genotype determination of steps (a) and (b); and
(d) marketing the antibody to health care providers or health care consumers for administration to the patient selected in step (c) for the treatment of a disease or disorder.

12-19. (canceled)

20. The method of claim 11, wherein the disease or disorder is selected from a neoplastic disease, an autoimmune disease, a microbial infection, and allograft rejection.

21. The method of claim 20, wherein the neoplastic disease is non-Hodgkin's lymphoma (NHL).

22. The method of claim 21, wherein the NHL is follicular lymphoma.

23. The method of claim 11, wherein the antibody is a monoclonal antibody, a polyclonal antibody or an Fc fusion protein.

24-25. (canceled)

26. The method of claim 11, wherein the marketing is accomplished by continuing medical education, peer-review journals, Internet, print advertising or direct sale calls.

27. The method of claim 11, wherein the health care providers are selected from the group consisting of physicians, nurses, hospitals and medical insurance providers.

28. (canceled)

29. A method of doing business, comprising:
(a) using the determination of a patient's FcγRIIIa V/V¹⁵⁸, FcγRIIa H/H¹³¹; FcγRIIIa V/F¹⁵⁸, FcγRIIa H/H¹³¹; FcγRIIIa F/F¹⁵⁸, FcγRIIa H/H¹³¹; FcγRIIIa V/V¹⁵⁸, FcγRIIa H/R¹³¹; FcγRIIIa V/V¹⁵⁸, FcγRIIa R/R¹³¹; FcγRIIIa V/F¹⁵⁸, FcγRIIa R/R¹³¹; FcγRIIIa F/F¹⁵⁸, FcγRIIa R/R¹³¹; FcγRIIIa V/F¹⁵⁸, FcγRIIa H/R¹³¹; or FcγRIIIa F/F¹⁵⁸, FcγRIIa H/R¹³¹ genotype to select an antibody for treatment of a disease or disorder; and
(b) marketing the antibody to health care providers or health care consumers for administration to the patient for treatment of the disease or disorder.

30-38. (canceled)

39. A method of selecting subjects for a clinical trial, said method comprising:
(a) determining the FcγRIIIa¹⁵⁸ and FcγRIIa¹³¹ genotype of the subjects having an optimal level of response to an antibody; and
(b) using subjects with the genotype selected in (a) in clinical trials with the antibody.

40-43. (canceled)

44. A method for generating or increasing revenue, said method comprising:
(a) optimizing a therapeutic antibody used to treat a disease or disorder in subjects with a FcγRIIIa V/V¹⁵⁸, FcγRIIa H/H¹³¹; FcγRIIIa V/F¹⁵⁸, FcγRIIa H/H¹³¹; FcγRIIIa F/F¹⁵⁸, FcγRIIa H/H¹³¹; FcγRIIIa V/V¹⁵⁸, FcγRIIa H/R¹³¹; FcγRIIIa V/V¹⁵⁸ FcγRIIa R/R¹³¹; FcγRIIIa V/F¹⁵⁸, FcγRIIa R/R¹³¹; FcγRIIIa F/F¹⁵⁸, FcγRIIa R/R¹³¹; FcγRIIIa V/F¹⁵⁸, FcγRIIa H/R¹³¹; or a FcγRIIIa F/F¹⁵⁸, FcγRIIa H/R¹³¹ genotype; and
(b) marketing the optimized antibody to health care providers or health care consumers with the disease or disorder.

45-51. (canceled)

52. A method of conducting a business, wherein:
a) said business markets one or more components of an antibody therapy for an ADCC-mediated disease or disorder in a patient to
i) a health care provider;
ii) a medical insurance provider; or
iii) a health care consumer; and
b) said marketing is different and depends upon the FcγRIIIa V/F¹⁵⁸ and the FcγRIIa H/R¹³¹ genotypes of said patient.

53. The business method of claim 52, wherein:
a) said business supplies one or more good(s) or service(s) related to said therapy to a health care provider;
b) said business supplies one or more good(s) or service(s) related to evaluation, selection, or reimbursement for said therapy to a medical insurance provider;
c) said business supplies one or more good(s) or service(s) related to said therapy to a health care consumer;
d) said one or more components of an antibody therapy includes an antibody therapeutic;
e) said antibody therapy is one or more of a monoclonal antibody, polyclonal antibody, or antibody Fc fusion protein therapy;
f) said one or more components of an antibody therapy includes a service related to administration of said antibody;
g) said ADCC-mediated disease or disorder is selected from a neoplastic disease, an autoimmune condition, a microbial infection, or an allograft rejection;
h) said FcγRIIIa V/F¹⁵⁸ and FcγRIIa H/R¹³¹ genotypes correlate with a similar response to treatment;
i) said FcγRIIIa V/F¹⁵⁸ and FcγRIIa H/R¹³¹ genotypes correlate with at least three categories with similar responses to treatment;
j) said FcγRIIIa V/F¹⁵⁸ and FcγRIIa H/R¹³¹ genotypes distinguish treatment responses of patient groups: V/V¹⁵⁸ H/H¹³¹ from F/F¹⁵⁸ R/R¹³¹ (Group-I from IX), V/V¹⁵⁸ H/H¹³¹ from F/F¹⁵⁸ H/R¹³¹ (Group-I from VI), V/V¹⁵⁸ H/F¹³¹ from V/F¹⁵⁸ R/R¹³¹ (Group-I from VIII), or V/V¹⁵⁸ H/H¹³¹ from V/F¹⁵⁸ H/R¹³¹ (Group-I from V);
k) said FcγRIIIa V/F¹⁵⁸ and FcγRIIa H/R¹³¹ genotypes of said patient are a factor in customizing treatment procedures of a therapy for said patient;
l) said FcγRIIIa V/F¹⁵⁸ and FcγRIIa H/R¹³¹ genotypes of said patient assist in selecting a preferred therapy for said patient;
m) said FcγRIIIa V/F¹⁵⁸ and FcγRIIa H/R¹³¹ genotypes of said patient assist in deciding against a therapy for said patient;
n) said FcγRIIIa V/F¹⁵⁸ and FcγRIIa H/R¹³¹ genotypes of said patient indicate greater marketing efforts to be applied;
o) said FcγRIIIa V/F¹⁵⁸ and FcγRIIa H/R¹³¹ genotypes of said patient indicate lesser marketing efforts to be applied;
p) said genotype-dependent responsiveness to therapy is gathered and used to selectively market or sell such information to one or more parties, including a health care provider, including a physician, nurse, hospital, a medical insurance provider, said patient, or employer;
q) said method further comprises determination of said FcγRIIIa V/F¹⁵⁸ and/or FcγRIIa H/R¹³¹ genotypes, which determination may be evaluation of nucleic acid or polypeptide;
r) said business markets to a medical insurer and differently markets treatment evaluation, treatment selection, or treatment reimbursement;
s) said business markets to a medical insurer and differently markets treatment evaluation, treatment selection, or treatment reimbursement, which different marketing may include different pricing, promotion, product placement, or genotype-dependent responsiveness forms of continuing medical education, advertisements, journal publications, sales efforts, internet marketing, or information provided to patients; or
t) said different marketing is supported by clinical trial data indicating different responsiveness to therapy.

54. The method of claim 52, wherein said method:
a) chooses a particular antibody useful for treatment of said ADCC-mediated disease or disorder based, in part, upon said FcγRIIIa and FcγRIIa genotypes and markets said chosen antibody;
b) selects a patient whose FcγRIIIa and FcγRIIa genotypes match with a preferred antibody to provide high responsiveness and markets said antibody to a health care provider;
c) selects a patient for treatment with a preferred antibody which depends, in part, upon said patient's FcγRIIIa and FcγRIIa genotypes and markets said antibody to a health care provider;
d) selects a patient for treatment with a selected antibody, based, in part, upon the patient's FcγRIIIa and FcγRIIa genotypes and markets said antibody to a health care provider; or
e) selects a preferred therapeutic antibody for treatment of a patient based, in part, upon the patients' FcγRIIIa and FcγRIIa genotypes and markets said antibody to a health care provider.
